(19) United States
(12) Patent Application Publication
(10) Pub. No.: US 2015/0335610 A1
(43) Pub. Date: Nov. 26, 2015

(54) PDK4 INHIBITOR AND USE THEREOF

(71) Applicants: THE KITASATO INSTITUTE, Tokyo (JP); THE UNIVERSITY OF TOKUSHIMA, Tokushima-shi, Tokushima (JP)

(72) Inventors: Satoshi OMURA, Tokyo (JP); Hirofumi NAKANO, Tokyo (JP); Kenzaburo YAMAJI, Tokyo (JP); Tsuyoshi YAMAMOTO, Tokyo (JP); Hiroshi KIDO, Tokushima-shi (JP); Junji CHIDA, Tokushima-shi (JP); Kazuhiko YAMANE, Tokushima-shi (JP)

(73) Assignees: THE UNIVERSITY OF TOKUSHIMA, Tokushima-shi, Tokushima (JP); THE KITASATO INSTITUTE, Tokyo (JP)

(21) Appl. No.: 14/391,753

(22) PCT Filed: Oct. 17, 2013
(86) PCT No.: PCT/JP2013/002500

§ 371 Date: Aug. 5, 2015

Related U.S. Application Data
(60) Provisional application No. 61/623,501, filed on Apr. 12, 2012.

(51) Int. Cl. A61K 31/352; A61K 8/34; A61K 31/343; A61K 8/40; A61K 8/49; A61K 31/11; A61K 31/277

(52) U.S. Cl. CPC A61K 31/352; A61K 31/11; A61K 8/347; A61K 31/277; A61K 8/40; A61K 8/498; A61K 31/343; A61K 8/4973; A61K 2800/40

(57) ABSTRACT

The purpose of the present invention is to provide a therapeutic agent or preventive agent for exacerbation of influenza, specifically to provide a novel pyruvate dehydrogenase kinase 4 (PDK4) inhibitor. The present invention relates to a PDK4 inhibitor, medical composition or cosmetic composition containing as the active ingredient a compound represented by any one of the following general formulas (I) through (III) and a pharmaceutically acceptable ester derivative thereof, or a pharmaceutically acceptable salt of the same.

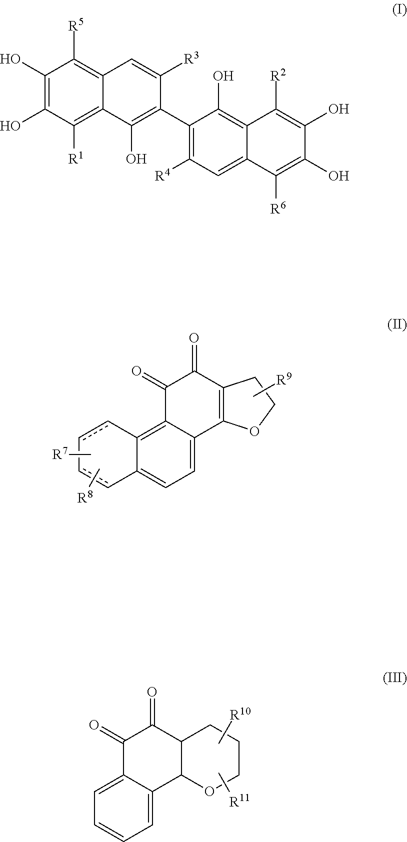

### TECHNICAL FIELD

The present invention relates to a novel compound that inhibits pyruvate dehydrogenase kinase 4 (hereinafter referred to as “PDK4”), pharmaceutically acceptable ester derivatives thereof, and pharmaceutically acceptable salts thereof. The present invention also relates to a method for treatment or prevention of diseases, wherein expression or activation of PDK4 is involved in development or aggravation of said diseases. More specifically, the present invention is directed to an agent for treatment or prevention of aggravation after influenza infection, anorexia, mitochondrial disease, a diseases or disorders involving decreased ATP production, diabetes or cancer. Alternatively, the present invention is directed to a method for treatment or prevention for these diseases or disorders. The present invention further relates to a cosmetic composition containing a novel PDK4-inhibitor compound, or a pharmaceutically acceptable ester derivatives thereof, or apharmaceutically acceptable salts thereof.

### BACKGROUND ART

A healthy adult infected with influenza virus can recover without becoming severe and acquire immunity against the infected virus. However, an elderly or a child infected with influenza virus may develop multiple organ failure (MOP) or influenza-associated encephalopathy (IAE), which can result in death. Today, anti-virus drugs developed in late 1990s such as neuraminidase inhibitors are administered to patients infected with influenza virus. However, in 2012, the result of large-scale analysis of clinical trial data is reported, where administration of oseltamivir (Tamiflu) or zanamivir (Relenza) certainly relieved initial symptoms (i.e. the symptoms of influenza subside about one day earlier), but had no effect on prevention of aggravation after infection (Non-Patent Literature 1).

A mitochondria localized enzyme, pyruvate dehydrogenase (hereinafter referred to as “PDH”) is a key enzyme in control of sugar metabolism, which is deactivated upon phosphorylation by PDK. In humans and mice, there are four types of PDK isozymes (PDK1 to PDK4). PDK4 is known to be associated with the development and aggravation of diabetes and cancer (see Non-Patent Literatures 2 and 3). Since PDH level is decreased due to overexpression of PDK in cancer and diabetes patients, PDK has drawn attention as a molecular target for cancer and diabetes drugs, and PDK inhibitor has been searched.

However, a compound that can inhibit PDK4 with IC50 of 100 μM or less has not been found yet. For example, though it is reported that drugs such as AZD7545, Compound K, and Novartis 3r inhibit PDK isozymes 1, 2, and 3 with IC50 of less than 1 μM order, these drugs adversely promote the activity of PDK4. Unlike PDK1 to PDK3, PDK4 exists in a semi-active state, which is assumed to be a reason that makes the development of PDK4 inhibitors difficult. A dichloroacetic acid is reported as a PDK4 inhibitor, however it could not be used for a medicine because of its weak inhibitory activity and severe side effects such as neurotoxicity (see Non-Patent Literature 4).

### CITATION LIST

### Non-Patent Literature

Non-Patent Literature 1: Published Online: 18 Jan. 2012 DOI: 10.1002/14651858.CD008965.pub3
Non-Patent Literature 2: Int. J. Cancer 2011: 128: 1001 to 1008
Non-Patent Literature 3: Biochem. J. (2009) 423: 243 to 252
Non-Patent Literature 4: J. Biol Chem. (2008) 283: 25305 to 25315

### SUMMARY OF INVENTION

An object of the present invention is to provide a novel PDK4-inhibitor compound having a stronger inhibiting activity than existing compounds.

The inventors Kido et al. have analyzed the pathogenic mechanism of aggravation of influenza, and reported that adenosine triphosphate (hereinafter, referred to as “ATP”) level reduces due to viral infection in the peripheral blood of patients accompanying IAE or MOF, and that they found a temperature-sensitive genetic polymorphism in a mitochondrial fatty acid metabolism enzyme carnitine palmitoyltransferase 2 (CPT2) (Mol Cell Biochem (2007) 299: 85 to 92; Hum Mutat (2008) 29: 718 to 727). The inventors infected 3-week-old mice with influenza virus to comprehensively analyze the expression levels of genes involved in the mitochondrial energy production system and found that following influenza virus infection, the PDK4 gene expression was induced accompanied with increased cytokine production and pyrexia. Based on these findings, the inventors presumed that an acute reduction of activity of mitochondrial function may cause systemic ATP depletion in aggravation of influenza-infected patients, and thus a PDK4 inhibitor could prevent the acute aggravation.

Since 1980s, the inventors Nakano and Omura et al. have discovered staurosporine and related substances thereof as a protein kinase (PK) inhibitor (J. Antibiotics (2009) 62: 17 to 26). According to the above presumption of Kido et al, the inventors searched for novel PDK4 inhibitors to provide a novel drug for preventing aggravation of influenza-infected patients. Although PDK is a Ser/Thr kinase, PDK is not inhibited by staurosporine that is recognized as a powerful universal kinase inhibitor. The structural analysis of the ATP-binding site of PDK4 revealed that staurosporine can not attach in the site. The inventors searched for new compounds that inhibits PDK4 at μM order from natural molecules that were smaller than staurosporine and finally found compounds of the present invention. By examining effect of the compounds of the present invention using a mouse model of influenza infection, the inventors confirmed that the compound of the present invention have a suppressive activity on aggravation such as loss of appetite, body weight loss and so on as well as on death, and thereby completed the present invention.

As described above, in the aggravated influenza-infected patients, the expression of the PDK4 gene is induced, the ATP level in the peripheral blood is reduced due to viral infection, and the mitochondrial fatty acid metabolism enzyme (CPT2) has a temperature-sensitive genetic polymorphism. Therefore, the PDK4 inhibitors of the present invention are considered to be also useful for the treatment of diseases associated with mutations in CPT or mitochondrial ATP-producing enzymes.

As above described, it is known that PDK4 is also associated with the development and aggravation of diabetes and cancer (see Int. J. Cancer (2011) 128: 1001 to 1008; and Biochem. J. (2009) 423: 243 to 252), and thus the PDK4 inhibitors of the present invention are also considered to be useful for treating diabetes and cancer.

One object of the present invention is to provide a therapeutic or preventive drug for aggravation of influenza. In another embodiment, one object of the present invention is to provide a novel PDK4 inhibitor. Further, the present invention aims to provide an agent for improving the mitochondrial function and loss of appetite, or to provide a therapeutic drug for diseases such as cancer or diabetes as well as a cosmetic that improves metabolism, comprising a novel PDK4 inhibitor as an active ingredient.

In one embodiment, the present invention relates to a PDK4 inhibitor, a pharmaceutical composition, or a cosmetic composition, comprising a compound represented by any one of the following formulaes (I) to (III) (hereinafter, collectively referred to as the “compound of the present invention”) or pharmaceutically acceptable ester derivatives thereof, or pharmaceutically acceptable salts thereof as an active ingredient:

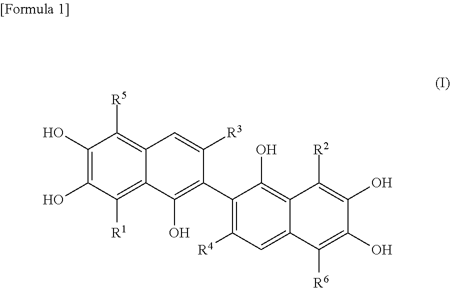

wherein, R¹ and R² can be the same or different and each represent a formyl group or a 2-carboxyphenyliminomethyl group, and R³ to R⁶ can be the same or different and each represents a linear or branched C1-6 alkyl group;

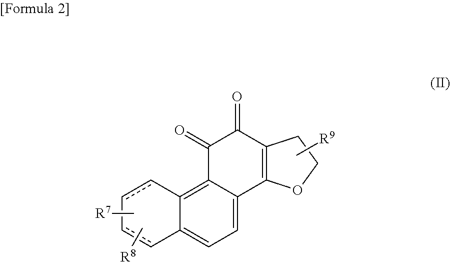

wherein, a bond represented by a solid line and a broken line represents a single bond or a double bond, R⁷ and R⁸ can be the same or different and each represents a linear or branched C1-6 alkyl group, and R⁹ represents a linear or branched C1-6 alkyl group;

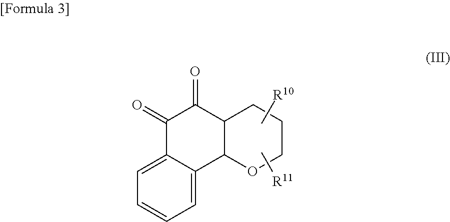

wherein, R¹⁰ and R¹¹ can be the same or different and each represents a linear or branched C1 to 6 alkyl group.

Specifically, the said pharmaceutical composition is a therapeutic or preventive drug for diseases or disorders whose development or aggravation is associated with or contributed by the expression or activation of PDK4. More specifically, diseases or disorders whose development or aggravation is associated with or contributed by the expression or activation of PDK4 include, but are not limited to, aggravation after influenza infection, loss of appetite, mitochondrial disease, or a disease or disorder associated with reduced ATP production, diabetes, or cancer.

Herein, the “linear or branched C1-6 alkyl group” refers to a linear or branched, saturated hydrocarbon group having 1 to 6 carbon atoms, and includes, for example, a methyl group, an ethyl group, a n-propyl group, an i-propyl group, a n-butyl group, a sec-butyl group, a t-butyl group, an isobutyl group, a pentyl group, an isopentyl group, a 2,3-dimethylpropyl group, a hexyl group, and a cyclohexyl group, which is preferably a C1-5 alkyl group, more preferably a methyl group, an ethyl group, a n-propyl group, an i-propyl group, a n-butyl group, a sec-butyl group, a t-butyl group, an isobutyl group, a pentyl group, an isopentyl group, or a 2,3-dimethylpropyl group, and even more preferably a C1-4 alkyl group such as a methyl group, an ethyl group, a n-propyl group, an i-propyl group, a n-butyl group, a sec-butyl group, a t-butyl group, and an isobutyl group. The linear or branched C1-6 alkyl group as represented by R³, R⁴ and R⁷ to R¹¹ is preferably a methyl group, an ethyl group, and a propyl group (such as a n-propyl group), more preferably a methyl group. The linear or branched C1-6 alkyl group as represented by R⁵ and R⁶ is preferably a n-propyl group, an i-propyl group, a n-butyl group, a sec-butyl group, a t-butyl group, and an isobutyl group, more preferably an i-propyl group, a sec-butyl group, a t-butyl group, and an isobutyl group, and most preferably an i-propyl group. For example, in formula (I), R¹ and R² are the same or different and each represents a formyl group or a 2-carboxyphenyliminomethyl group, R³ and R⁴ are the same or different and each represents a methyl group, an ethyl group, or a propyl group (such as a n-propyl group) (preferably, a methyl group), and R⁵ and R⁶ are the same or different and each represents a n-propyl group, an i-propyl group, a n-butyl group, a sec-butyl group, a t-butyl group, and an isobutyl group (preferably, an i-propyl group, a sec-butyl group, a t-butyl group, and an isobutyl group, more preferably an i-propyl group). Also, in formula (II), a bond represented by a solid line and a broken line represents a single bond or a double bond, R⁷ and R⁸ can be the same or different and each represents a methyl group, an ethyl group, or a propyl group (such as a n-propyl group) (preferably, a methyl group), and R⁹ represents a methyl group, an ethyl group, or a propyl group (such as a n-propyl group) (preferably a methyl group). In formula (III), R¹⁰ and R¹¹ can be the same or different and each represents a methyl group, an ethyl group, or a propyl group (such as a n-propyl group) (preferably, a methyl group).

In a preferable embodiment, the compound of the present invention represented by formula (II) is represented by the following (II-a) or (II-b). In the following formulae (II-a) and (II-b), when a bond represented by a solid line and a broken line is a double bond, R⁸ does not exist.

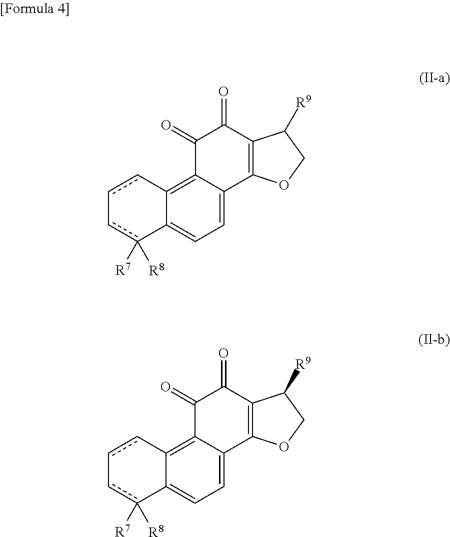

Also, in a preferable embodiment, the compound of the present invention represented by formula (III) is represented by the following (III-a).

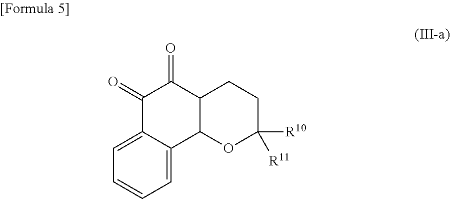

Particularly, the inventors verified that KIS7, KIS28, KIS37, KIS116 and KIS24 having the following formulae inhibit PDK4 and have potent preventive activities on acute aggravation of influenza from enzyme inhibition assays and animal experiments.

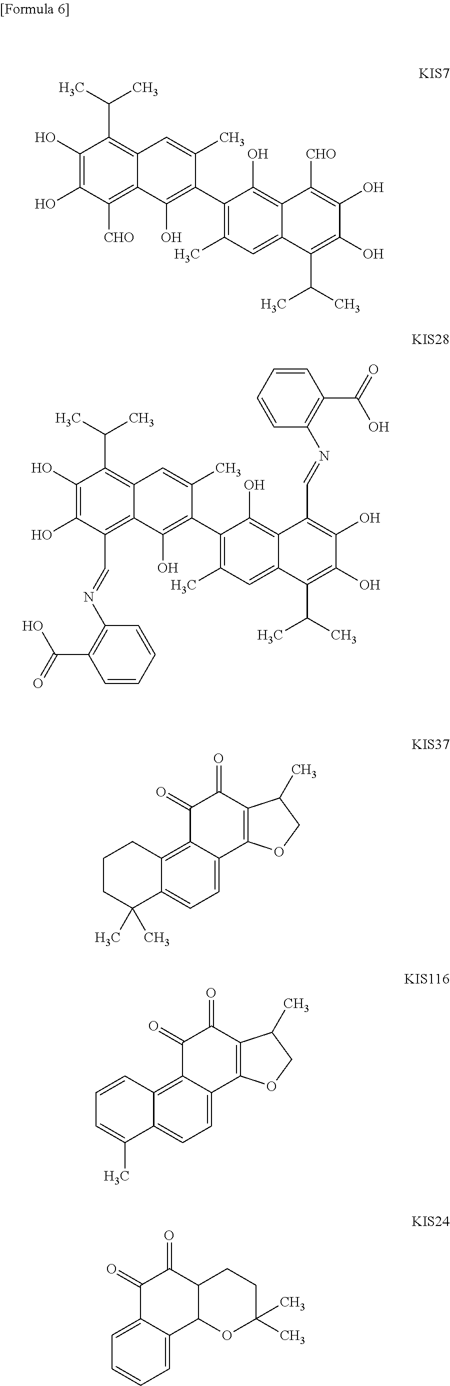 [SMILES: Cc1cc2c(C(C)C)c(O)c(O)c(C=O)c2c(O)c1-c1c(C)cc2c(C(C)C)c(O)c(O)c(C=O)c2c1O ; Cc1cc2c(C(C)C)c(O)c(O)c(/C=N/c3ccccc3C(=O)O)c2c(O)c1-c1c(C)cc2c(C(C)C)c(O)c(O)c(/C=N/c3ccccc3C(=O)O)c2c1O ; CC1COC2=C1C(=O)C(=O)c1c2ccc2c1CCCC2(C)C ; Cc1cccc2c3c(ccc12)C1=C(C(=O)C3=O)C(C)CO1 ; CC1(C)CCC2C(=O)C(=O)c3ccccc3C2O1]

In addition to the compounds described above, the present invention encompasses pharmaceutically acceptable ester derivatives of those compounds. Here, the “pharmaceutically acceptable ester derivative” means an ester compound having a group which is metabolized to generate the above compounds in human body, that can be administered into the human as a medicine. Herein, ester encompasses an amide-linked compound as well as an ester-linked compound. Ester may generate an active compound through degradation by esterase in vivo. Examples of the ester include a substituted or an unsubstituted lower alkyl ester, lower alkenyl ester, lower alkylamino lower alkyl ester, acylamino lower alkyl ester, acyloxy lower alkyl ester, aryl ester, aryl lower alkyl ester, amide, lower alkylamide, and hydroxylamide. The ester is preferably a propionic acid ester or an acyl ester. Herein, the “compounds represented by formulae (I) to (III)” or the “compound of the present invention” encompasses pharmaceutically acceptable ester derivatives of the compounds represented by formulae (I) to (III) or of the compound of the present invention as long as such derivatives are evidently not suitable, even not explicitly specified.

The “pharmaceutically acceptable salt” refers to a salt formed by binding the compound of the present invention or pharmaceutically acceptable ester derivatives thereof to an inorganic or organic base or acid, which can be administered into the body as a medicine. Such salts are described in, for example, Berge et al., J. Pharm. Sci. 66: 1 to 19 (1977). For example, when the compound of the present invention or pharmaceutically acceptable ester derivatives thereof has an acidic group such as a carboxylic acid group, the compound or pharmaceutically acceptable ester derivatives thereof can form a salt with alkali metal and alkaline earth metal such as lithium, sodium, potassium, magnesium, and calcium; a salt with an amine such as ammonia, methylamine, dimethylamine, trimethylamine, dicyclohexylamine, tris(hydroxymethyl)aminomethane, N,N-bis(hydroxyethyl)piperazine, 2-amino-2-methyl-1-propanol, ethanolamine, N-methylglucamine, and L-glucamine; or a salt with a basic amino acid such as lysine, δ-hydroxylysine, and arginine. When the compound of the present invention or pharmaceutically acceptable ester derivatives thereof has a basic group, examples of the salt include a salt formed with a mineral acid such as hydrochloric acid, hydrobromic acid, sulfuric acid, nitric acid, and phosphoric acid; a salt formed with an organic acid such as methanesulfonic acid, benzenesulfonic acid, paratoluenesulfonic acid, acetic acid, propionate, tartaric acid, fumaric acid, maleic acid, malic acid, oxalic acid, succinic acid, citric acid, benzoic acid, mandelic acid, cinnamic acid, lactic acid, glycolic acid, glucuronic acid, ascorbic acid, nicotinic acid, and salicylic acid; or a salt formed with an acidic amino acid such as aspartic acid and glutamic acid. The compound of the present invention also encompasses a hydrate or solvate of the compound of the present invention and a hydrate or solvate of a pharmaceutically acceptable salt of the compound of the present invention. Further, herein the “compounds represented by formulae (I) to (III)” or the “compound of the present invention” also encompasses pharmaceutically acceptable salts, hydrates, and solvates of the compounds represented by formulae (I) to (III) or pharmaceutically acceptable ester derivatives thereof; pharmaceutically acceptable hydrates or solvates of pharmaceutically acceptable salts of the compounds represented by formulae (I) to (III) or pharmaceutically acceptable ester derivatives thereof; pharmaceutically acceptable salts, hydrates, and solvates of the compound of the present invention or pharmaceutically acceptable ester derivatives thereof; and a hydrate or solvate of a pharmaceutically acceptable salt of the compound of the present invention or pharmaceutically acceptable ester derivatives thereof.

Because the compound of the present invention optionally has asymmetric carbon, optical isomers may exist. The compound of the present invention may be either a dextrorotatory (+) compound or a levorotatory (−) compound, or a mixture of these isomers such as a racemate. Also, unless otherwise noted, the compound of the present invention also encompasses any tautomer or geometrical isomer (such as E-form and Z-form).

In the present specification, the PDK4 inhibitor is not particularly limited as long as it is a drug used to inhibit PDK4. Preferably, the PDK4 inhibitor of the present invention is provided as a pharmaceutical composition. Such a pharmaceutical composition can be used for the treatment or prevention of diseases or disorders whose development or aggravation is associated with or contributed by the expression or activation of PDK4, including aggravation after influenza infection (including body weight loss, eating disorder, and/or water intake disorder after influenza infection), loss of appetite, mitochondrial disease, a disease or disorder associated with reduced ATP production, diabetes, or cancer. A mitochondrial disease means a disease or disorder based on mutations occurring in mitochondrial ATP synthases, and includes pyruvate dehydrogenase deficiency and MELAS. Examples of the disease or disorder associated with reduced ATP production include a disease or disorder based on mutated carnitine palmitoyltransferase.

The type of the pharmaceutical composition is not particularly limited, and its dosage forms include a tablet, a capsule, a granule, a powder, a syrup, a suspension, a suppository, an ointment, a cream, a gel, a patch, an inhalant, and an injection. These preparations can be prepared in accordance with a conventional method. When the pharmaceutical composition is a liquid preparation, it can be provided in such a form that is dissolved or suspended in water or other suitable solvents before use. A tablet and a granule can be subjected to coating by a well-known method. When the pharmaceutical composition is an injection, it is prepared by dissolving the compound of the present invention in water or, if necessary, physiological saline or a glucose solution, and a buffer and a preservative may also be added. The pharmaceutical composition is provided in any dosage form for oral or parenteral administration. For example, the pharmaceutical composition can be prepared as an orally administered pharmaceutical composition in the forms such as a granule, a fine granule, a powder, a hard capsule, a soft capsule, a syrup, an emulsion, a suspension, or a liquid, or as a parenterally administered pharmaceutical composition in the forms such as an injection for intravenous, intramuscular, or subcutaneous administration, a drip infusion, a transdermal agent, a transmucosal agent, a nasal drop, an inhalant, and a suppository. It is also possible to prepare an injection, a drip infusion, and the like in a powder form such as a freeze-dried form and dissolve them in suitable aqueous media such as physiological saline before use.

### Advantageous Effects of Invention

As will be demonstrated below, the present invention revealed that the compound of the present invention showed a novel enzyme inhibitory activity and was efficacious in an animal model. The compound of the present invention is the first reported drug that inhibits PDK4 at μM order. Specifically, the compound of the present invention was confirmed to exert a potent effect at a dose of less than 1/100 of the only existing drug, dichloroacetic acid. Hence, the compound of the present invention provides a novel PDK4 inhibitor having a potent inhibitory activity. More specifically, when the compound of the present invention was administered to a mouse model of influenza infection, it was confirmed to protect the mouse against body weight loss, and furthermore against death. Further, it was observed that the food and water intake recovered to close to the non-infected mouse level and that parameters such as the ATP level improved in biochemical analysis. The compound of the present invention proved to have a preventive action against acute aggravation of influenza infection. Accordingly, the compound of the present invention can treat or prevent aggravation of influenza, particularly body weight loss, eating disorder, and/or water intake disorder. Specifically, since the compound of the present invention can suppress body weight loss due to influenza infection, it can be used as a suppressant for body weight loss due to influenza infection. Also, since the compound of the present invention can suppress a decrease of water intake due to influenza infection, it can be used as a suppressant for a decrease of water intake due to influenza infection. The compound of the present invention suppressed a decrease of food intake. KIS7 has a suppressing effect on a decrease of food intake due to influenza infection, the compound of the present invention can be used as a suppressant for a decrease of food intake due to influenza infection. The compound of the present invention is effective in treating or preventing diseases or disorders dependent on reduced ATP levels. The compound of the present invention not only prevents aggravation due to influenza infection, but also is expected to be effective in diseases to be treated by inhibition of PDK4, and considered to be effective for treating or preventing such diseases. The compound of the present invention is also expected to be effective as a cosmetic by improving cellular metabolism caused by the activation of mitochondrial function, and considered to be useful also as a cosmetic.

### BRIEF DESCRIPTION OF DRAWINGS

FIG. 1 illustrates the mechanism of acute aggravation by influenza infection that is the results of analysis, and potential prevention of aggravation by a PDK4 inhibitor.

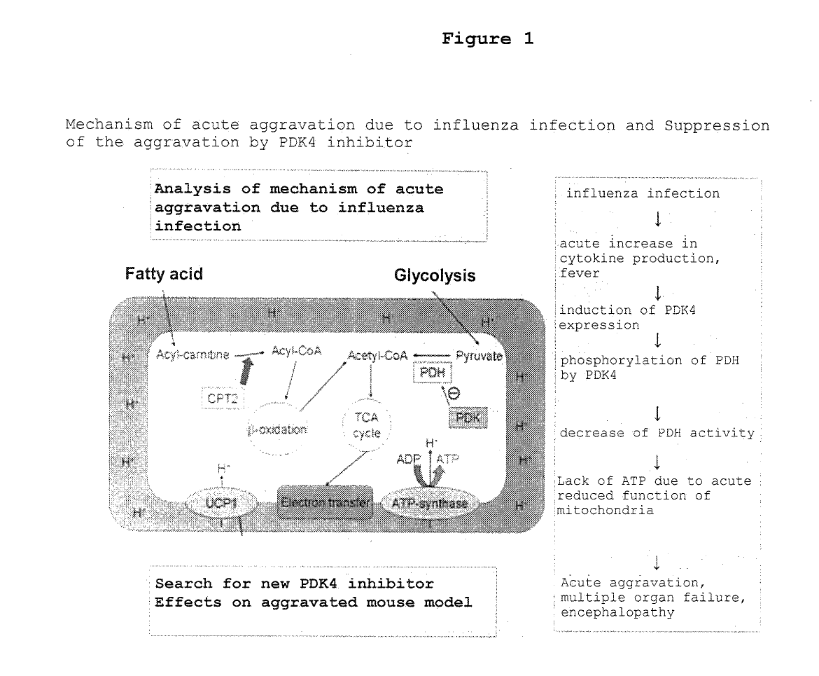

FIG. 2 illustrates that only PDK4 is in a semi-active state among other PDK isozymes. In the PDK2-ADP structure, (i) disordered C-terminal tails and (ii) a closed conformation that is closed active-site clefts (the T state) are observed. An active intermediate open conformation (the R′ state) with partially ordered cross-tails and open active-site clefts exists in the structures of human apo-PDK2, apo-PDK1, and PDK4-ADP. An active open conformation (the R state) with fully ordered cross-tails and open active-site clefts exists in the structures of human apo-PDK3-L2, PDK3-L2-ADP, PDK3-L2-ATP(1Y8P), PDK2-L2(3CRK), and PDK2-L2-(AMP-PNP)(3CRL).

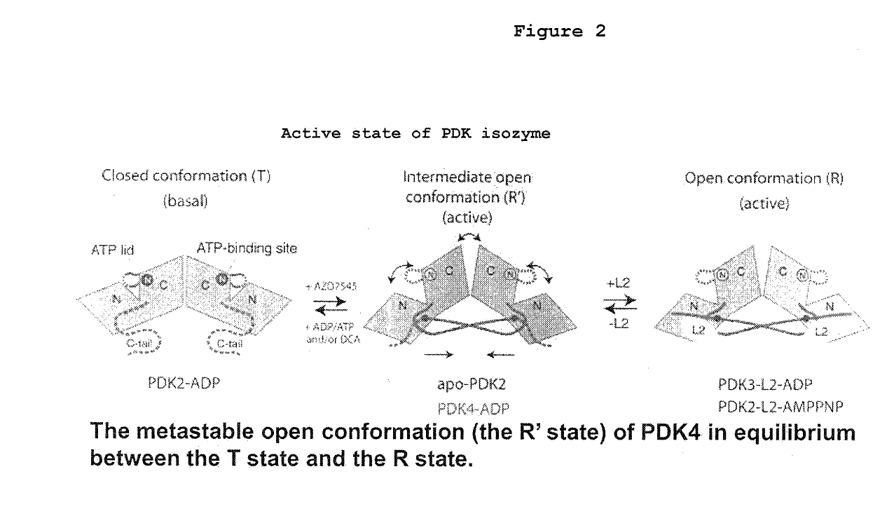

FIG. 3 shows the structures and PDK4 inhibitory activities of KIS7 and KIS28.

FIG. 4 is a table comparing activity among KIS7, KIS37, KIS116, KIS24, and existing PDK inhibitors. From the left, the compound used (Compound), the inhibitory activity on PDH phosphorylation by PDK1 (PDHK1), the inhibitory activity on PDH phosphorylation by PDK2 (PDHK2), and the inhibitory activity on PDH phosphorylation by PDK4, are shown in this order. The inhibitory activity on PDH phosphorylation by each PDK is expressed as the 50% inhibitory concentration (IC50) (μM).

FIG. 5 illustrates the schedule for a study of administration of KIS7, KIS28, KIS37, KIS116 and KIS24 to the mouse model of influenza infection (administered until Day 7).

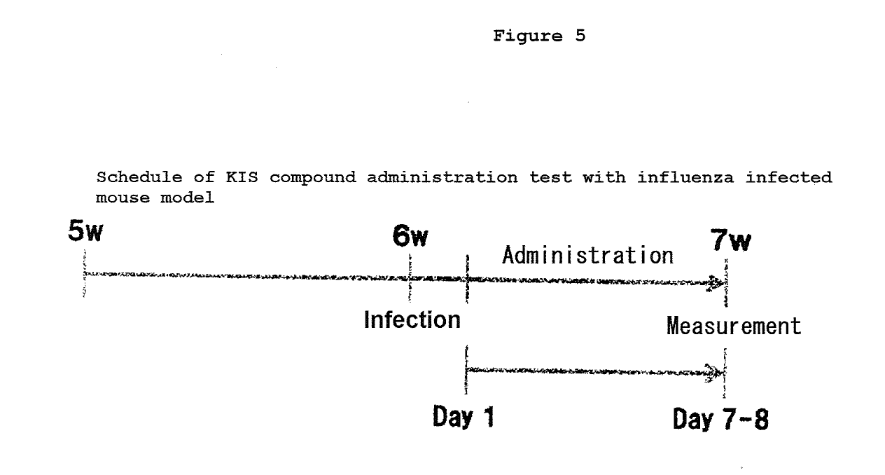

FIG. 6 A graph showing the changes in the body weight of the mouse model of influenza infection. The circles indicates the changes in the body weight of influenza-infected mice which were intraperitoneally administered KIS7 in 5% DMSO in physiological saline at 2.8 mg/kg/day. The controls were non-influenza-infected mice intraperitoneally administered 5% DMSO in physiological saline (diamond shapes); influenza-infected mice intraperitoneally administered 5% DMSO in physiological saline (squares); and influenza-infected mice intraperitoneally administered dichloroacetic acid (DCA) in 5% DMSO in physiological saline at 56 mg/kg/day (triangles). The vertical axis indicates the mouse body weight (g) and the horizontal axis indicates the number of days after infection (the initial infection day is designated as Day 0).

FIG. 7 Graphs showing the changes in water intake (graph on the left) and food intake (graph on the right) of the mouse model of influenza infection. The circle indicates the changes in food and water intake of influenza-infected mice which were intraperitoneally administered KIS7 in 5% DMSO in physiological saline at 2.8 mg/kg/day. Controls were non-influenza-infected mice intraperitoneally administered 5% DMSO in physiological saline (diamond shapes); influenza-infected mice intraperitoneally administered 5% DMSO in physiological saline (squares); and influenza-infected mice intraperitoneally administered dichloroacetic acid (DCA) in 5% DMSO in physiological saline at 56 mg/kg/day (triangles). The vertical axis in the right graph indicates the amount of water consumed by mice (g/mouse) and that in the left graph indicates the amount of food consumed by mice (g/mouse). In both graphs, the horizontal axes indicate the number of days after infection (the initial infection day is designated as Day 0).

FIG. 8 Graphs showing the results of each measured blood parameter in the blood obtained from the neck of mice at seven days after influenza virus infection. The blood parameters were: glucose level (mg/dL) (upper left graph), lactic acid level (mM) (upper right graph), β-hydroxybutyric acid (mM) (lower left graph), and ATP level (mM) (lower right graph). In each graph, the horizontal axis shows, from the left: non-influenza-infected mice intraperitoneally administered 5% DMSO in physiological saline; influenza-infected mice intraperitoneally administered 5% DMSO in physiological saline; influenza-infected mice intraperitoneally administered KIS7 in 5% DMSO in physiological saline at 2.8 mg/kg/day; and influenza-infected mice intraperitoneally administered dichloroacetic acid (DCA) in 5% DMSO in physiological saline at 56 mg/kg/day. In each graph, the vertical axis indicates the value of each parameter: blood glucose level (mg/dL) (upper left graph), lactic acid level (mM) (upper right graph), β-hydroxybutyric acid (mM) (lower left graph), and ATP level (mM) (lower right graph)).

FIG. 9 Graphs showing the ATP level in mouse tissue at seven days after influenza virus infection. The graphs show, from the left, heart, liver and muscle. In each graph, the horizontal axis shows, from the left: non-influenza-infected mice intraperitoneally administered 5% DMSO in physiological saline, influenza-infected mice given intraperitoneally administered 5% DMSO in physiological saline; influenza-infected mice intraperitoneally administered KIS7 in 5% DMSO in physiological saline at 2.8 mg/kg/day; and influenza-infected mice given intraperitoneally administered dichloroacetic acid (DCA) in 5% DMSO in physiological saline at 56 mg/kg/day. In each graph, the vertical axis indicates the ATP concentration in each tissue (μmol/g wet tissue).

FIG. 10 A graph showing the PDH enzyme activity in mouse liver tissue at seven days after influenza virus infection. The horizontal axis shows, from the left: non-influenza-infected mice intraperitoneally administered 5% DMSO in physiological saline; influenza-infected mice intraperitoneally administered 5% DMSO in physiological saline; influenza-infected mice intraperitoneally administered KIS7 in 5% DMSO in physiological saline at 2.8 mg/kg/day; and influenza-infected mice intraperitoneally administered dichloroacetic acid (DCA) in 5% DMSO in physiological saline at 56 mg/kg/day. The vertical axis indicates the PDH activity (ΔmOD/minute) in the liver.

FIG. 11 A graph showing the survival rate of the KIS7 administration group during 14 days after influenza infection. The vertical axis indicates the survival rate (%) and the horizontal axis indicates the number of days after infection (the initial infection day is designated as Day 0). Circles indicate Influenza-infected mice intraperitoneally administered KIS7 in 5% DMSO in physiological saline at 2.8 mg/kg/day. As controls, diamonds indicates non-influenza-infected mice intraperitoneally administered 5% DMSO in physiological saline, and squares indicate influenza-infected mice intraperitoneally administered 5% DMSO in physiological saline.

FIG. 12 The structure of KIS24 and a graph showing the survival rate of the KIS24 administration group during 14 days after influenza infection. In the graph, the vertical axis indicates the survival rate (%) and the horizontal axis indicates the number of days after infection (the initial infection day is designated as Day 0). Circles indicate Influenza-infected mice intraperitoneally administered KIS24 in 5% DMSO in physiological saline at 1.3 mg/kg/day. As controls, diamonds indicate non-influenza-infected mice intraperitoneally administered 5% DMSO in physiological saline, and squares indicate influenza-infected mice intraperitoneally administered 5% DMSO in physiological saline. Triangles indicate influenza-infected mice intraperitoneally administered dichloroacetic acid (DCA) in 5% DMSO in physiological saline at 56 mg/kg/day.

FIG. 13 The structures of KIS37 and KIS116 and a graph showing the survival rate during 14 days after influenza infection in the administration group. In the graph, the vertical axis indicates the survival rate (%) and the horizontal axis indicates the number of days after infection (the initial infection day is designated as Day 0). Circles indicates influenza-infected mice intraperitoneally administered KIS24 in 5% DMSO in physiological saline at 1.3 mg/kg/day. As controls, diamonds indicate non-influenza-infected mice intraperitoneally administered 5% DMSO in physiological saline, and squares indicate influenza-infected mice intraperitoneally administered 5% DMSO in physiological saline.

FIG. 14 Pictures showing the actions of KIS7, KIS37 and KIS24 on anchorage-independent cancer cell growth. KIS7, KIS37 and KIS24 inhibited the colony formation of cancer cells HeLaS3 in soft agar at 3 μM, which is of the same order as the inhibition of PDK4. Also, KIS7, KIS24, KIS37 and KIS 116 suppressed the anchorage-independent sphere growth of cells cancerated by Ras at μM to 10 μM order. Meanwhile, KIS7, KIS37 and KIS116 did not inhibit anchorage-independent growth of normal cells even at the order of several tens of μM.

### DESCRIPTION OF EMBODIMENTS

The compound of the present invention can be synthesized by appropriately adopting a chemical synthesis method well known to those skilled in the art, using a commercially available compound as a starting material.

The pharmaceutical composition of the present invention can be formulated by a conventional method using a common pharmaceutically acceptable carrier. In preparing an orally administered solid preparation, an excipient and, if necessary, a binder, a disintegrant, a lubricant and the like are added to a primary agent, and a solution, a granule, a powder, a capsule and the like are prepared by a conventional method. In preparation of an injection, in necessary, a pH adjustment agent, a buffer, a stabilizer, a solubilizing agent and the like are added to a primary agent, and an injection for subcutaneous or intravenous administration is prepared by a conventional method.

According to another embodiment, the present invention relates to a method for treatment or prevention of a disease or disorder whose development or aggravation is associated with the expression or activation of PDK (particularly, PDK4), comprising administering an effective amount of the compound of the present invention to a patient in need thereof. Alternatively, the present invention relates to a use of the compound of the present invention for the treatment or prevention of a disease or disorder whose development or aggravation is associated with PDK (particularly, PDK4).

The compound and pharmaceutical composition of the present invention can be administered in an oral dosage form or a parenteral dosage form such as an injection and a drip infusion. When the compound is administered to a mammal and the like, it can be orally administered in the form of a tablet, a powder, a granule, a syrup and the like. Alternatively, the compound can be administered parenterally in the form of an injection or a drip infusion. The dose can be appropriately set according to the severity of symptoms, age, body weight, sex, administration route, dosage form, responsiveness to a drug, type of disease and the like. For example, the compound of the present invention is normally administered to an adult at 50 to 500 mg per day once or in divided doses daily.

### Examples

Hereinafter, the present invention will be specifically described with reference to Examples, however, the present invention is not limited thereto. It should be noted that all documents cited throughout the present application are incorporated herein by reference in their entirety. Also, the present application claims priority to U.S. Provisional Application No. U.S. 61/623,501. The contents of U.S. Provisional Application No. U.S. 61/623,501, to which the present application claims priority, are wholly incorporated herein by reference in their entirety.

### Example 1

### Measurement of PDK-Inhibitory Activity

### (1) Preparation of Test Substances and Solutions Thereof

Test substances KIS7 (gossypol) and KIS24 (β-lapachone) were purchased from Enzo Life Sciences, Inc. (USA); KIS37 (cryptotanshinone) was purchased from Abcam plc. (USA); KIS116 (dihydrotanshinone I) was purchased from Sigma-Aldrich Co. LLC. (USA); KIS28 was purchased from Namiki Shoji Co., Ltd. (Japan); and the positive control dichloroacetic acid (DCA) was purchased from Wako Pure Chemical Industries, Ltd. (Japan).

The test substances (KIS7, KIS28, KIS24, KIS37, and KIS116) and positive control (dichloroacetic acid: DCA) were dissolved in dimethyl sulfoxide (DMSO) and then diluted to prepare solutions that were 100 times concentrated solution of the test concentration.

### (2) Measurement of the PDK2 and PDK4-Inhibitory Activities by Off-Chip Mobility Shift Assay

The PDK2 and PDK4-inhibitory activities were determined by measuring the phosphorylation of the E1 subunit of PDH in the presence of 100 μM ATP.

1) 5 μL of 4-fold concentrated solutions of test substances, 5 μL of 4-fold concentrated solutions of substrates (recombinant human PDH)/ATP/metal, and 10 μL of 2-fold concentrated solutions of human recombinant PDK were prepared with an assay buffer (20 mM HEPES, 0.01% Triton X-100, 2 mM DTT, pH 7.5) and then were mixed in the wells of a polypropylene 384-well plate, followed by reactions for five hours at room temperature.

2) Reactions were terminated by adding 60 μL of Termination Buffer (QuickScout Screening Assist MSA; Carna Biosciences, Inc.).

3) Peptide substrates and phosphorylated peptides in the reaction solutions were separated by LabChip3000 system (Caliper Life Science) and quantified (gel shift assay). The phosphorylation activity was assessed by product ratios (P/(P+S)) calculated from the peptide substrate peak height (S) and the phosphorylated peptide peak height (P).

4) The inhibitory percentage was calculated as follows. Assuming the phosphorylation activity in a well containing all reaction components as 0% inhibition and the phosphorylation activity in the absence of enzyme as 100% inhibition, the inhibitory percentage was calculated from the phosphorylation activity obtained for each test substance well.

### (3) Results

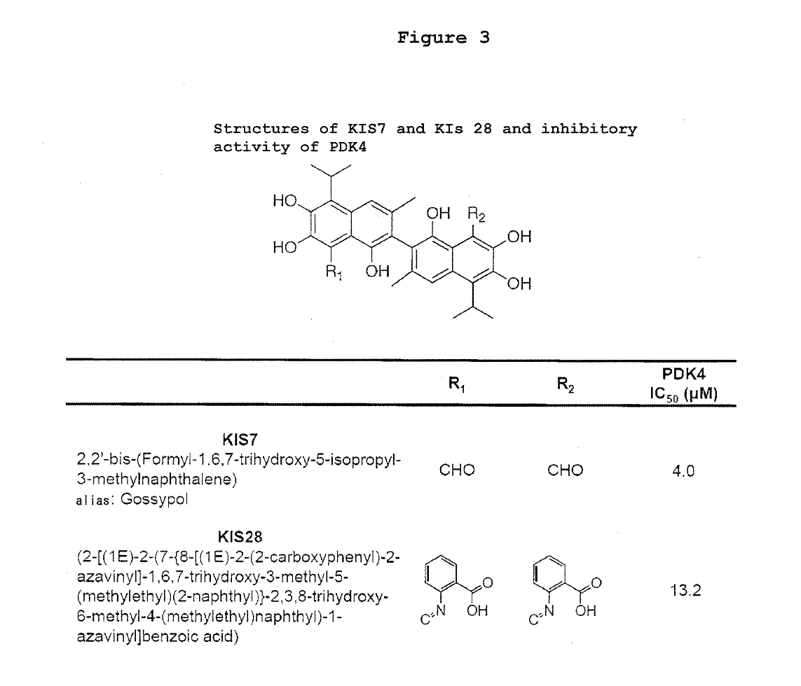

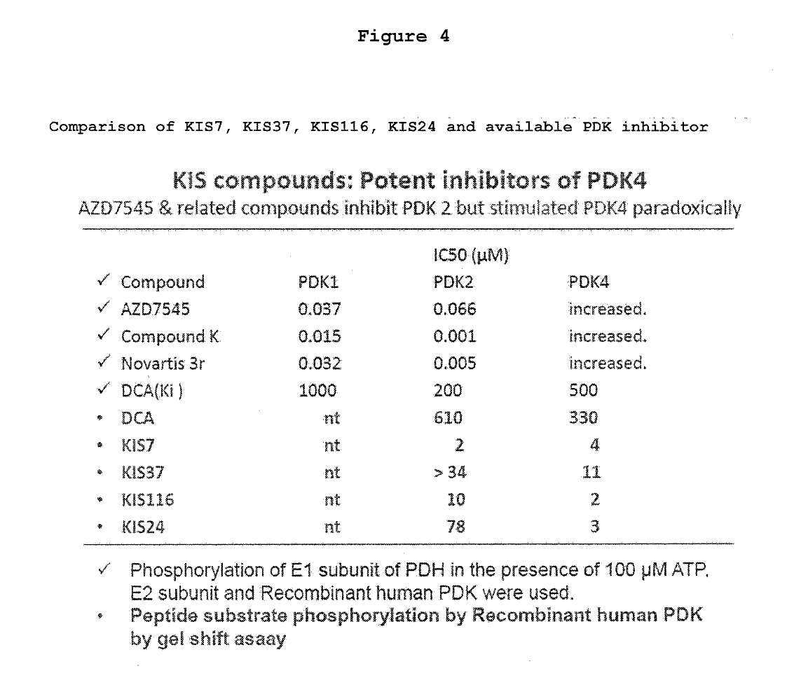

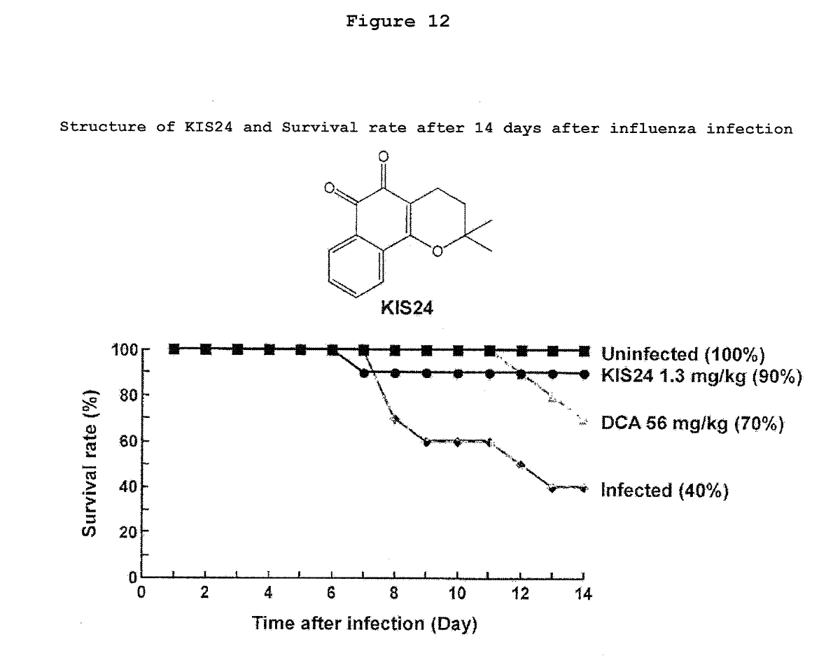

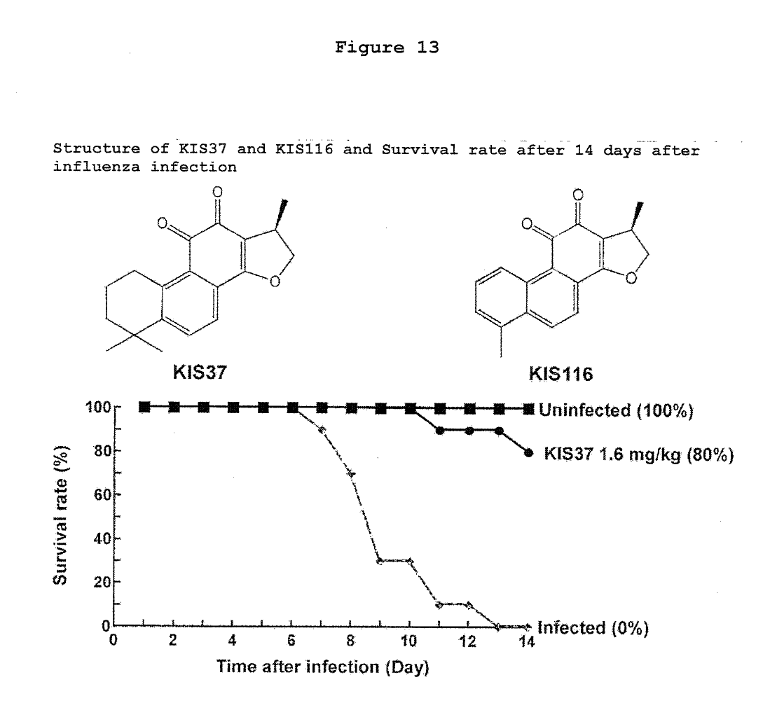

The PDK4-inhibitory activities of KIS7 and KIS28 are shown in FIG. 3. The results of comparing the PDK2 and PDK4-inhibitory activities between KIS7 and known PDK inhibitory substances are shown in FIG. 4. It was shown that while AZD7545, Compound K, and Novartis 3r activated PDK4, KIS7 inhibited PDK4. In addition, KIS7 successfully inhibited PDK4 at a far lower dose as compared with DCA and Radicicol. Also, the PDK2 and PDK4-inhibitory activities of KIS24, KIS37 and KIS116 are shown in FIG. 12 (KIS24) and FIG. 13 (KIS37 and KIS116).

As shown in said Figures, KIS7, KIS28, KIS24, KIS37 and KIS116 exhibited potent PDK4-inhibitory activities as 4.0 μM, 13.2 μM, 3 μM, 11 μM and <4 μM, respectively.

### Example 2

### Effects of KIS7 in the Mouse Model of Influenza Infection (7 Days Administration)

### (1) Mouse, Influenza Virus, and Reagent

Five-week-old female C57BL/6J mice (Japan SLC, Inc.) were purchased, and were anesthetized by intramuscular injection (a mixture of Ketalar 62.5 mg/kg and Celactal 12.5 μg/kg) in the sixth week (body weight: 16.4 to 18.1 g), followed by nasal infection with the influenza virus A/Puerto Rico 8/34 strain (influenza A/PR8/8/34 strain) suspension at 10 PFU/20 μL/mouse. A non-infected group mice were transnasally administered solvent of virus suspension, physiological saline (Otsuka Pharmaceutical Co., Ltd.) at 20 μL/mouse. The day of infection was designated as Day 0 (Pre-0). KIS7 in a solution of 5% DMSO (solvent) diluted in physiological saline was intraperitoneally administered at a dose of 2.8 mg/kg/day twice per day from the day after infection (Day 1) to Day 7. In each experiment, mice were grouped into the following four groups, and 10 mice were used per group (five mice per cage):

1) A group of non-infected mice given intraperitoneal administration of 5% DMSO in physiological saline,
2) A group of infected mice given intraperitoneal administration of 5% DMSO in physiological saline,
3) A group of infected mice given intraperitoneal administration of KIS compound (KIST) in 5% DMSO in physiological saline at 2.8 mg/kg/day, and
4) As a comparison, a group of infected mice given intraperitoneal administration of dichloroacetic acid (DCA) in 5% DMSO in physiological saline at 56 mg/kg/day.

### (2) Measurement of Body Weight, Food and Water Intake Amount

Body weight was individually measured once per day. The amount of food and water intake were measured by measuring the amount of food and water in a cage containing five mice per day and dividing the changed amount per cage by five to calculate the average intake amount per mouse.

### (3) Measurement of Biochemical Parameters in the Peripheral Blood

Blood was collected from the neck of five mice from each group on Day 7 after influenza virus infection to measure each parameter.

### (3-1) Measurement of the Blood Glucose Level

The blood glucose level was measured by Medisafe® mini GR-102 (Terumo Corpolation Japan), using several drops of blood in accordance with the operation manual provided by the manufacturer. The measurement principle is as follows: blood absorbed by the tip of the chip is developed on a test strip containing glucose oxidase, which convert glucose in the blood into hydrogen peroxide and gluconic acid. The generated hydrogen peroxide reacts with 4-aminoantipyrine and N-ethyl-N-(2-hydroxy-3-sulfopropyl)-m-toluidine contained in the reaction test unit by peroxidase to produce quinone dyes. By colorimetrically quantifying the resulting reddish purple coloration, the amount of glucose in the blood was calculated.

### (3-2) Measurement of the Lactic Acid Level

The lactic acid level was measured by Lactate Pro LT-1710 (Arkray Inc.), using several drops of blood in accordance with the operation manual provided by the manufacturer. The measurement principle is as follows: when blood is supplied to the electrode, an electron carrier potassium ferricyanide (oxidized form) in the reaction layer dissolves and produces potassium ferrocyanide (reduced form) by enzymatic reaction with lactate oxidase (LOD). Subsequently, a fixed voltage is applied to the electrode to oxidize potassium ferrocyanide, and the oxidation current generated thereby is measured. The generated oxidation current is converted to the amount of potassium ferrocyanide produced, i.e., the lactic acid concentration, whereby the lactic acid level was calculated.

### (3-3) Measurement of β-Hydroxybutyric Acid

As the representative ketone body in the blood, the β-hydroxybutyric acid level was measured. The β-hydroxybutyric acid was measured by Precision Xceed (Abbott Japan Co. Ltd), using several drops of blood in accordance with the operation manual provided by the manufacturer. The measurement principle is as follows: when blood is dropped on the electrode, β-hydroxybutyric acid (β-OHB) in the blood reacts with β-hydroxybutyric acid dehydrogenase in the electrode to generate a weak electrical current via an electron carrier. Since the current intensity depends on the concentration of β-OHB in the dropped blood, the β-hydroxybutyric acid level was calculated by measuring the electric current thus generated.

### (3-4) Measurement of the ATP Level

The ATP level was measured by extracting ATP from blood and using the luciferase reaction, by using AMERIC-ATP kit (Applied Medical Enzyme Research Institute Corporation) in accordance with the operation manual provided by the manufacturer.

### (4) Measurement of the ATP Level in Each Mouse Tissue

Using AMERIC-ATP kit (Applied Medical Enzyme Research Institute Corporation), the ATP level was measured by extracting ATP from each tissue and by the luciferase reaction in accordance with the operation manual provided by the manufacturer. Specifically, the total mass of the heart, approximately half mass of the brain and liver, and the total mass of the muscle part of the right hind leg as muscle were excised from mice on Day 7 after influenza virus infection and pulverized by a homogenizer (ULTRA-TURRAX T25 Digital: IKA Japan) in ATP extraction solutions. Subsequently, the obtained homogenized solutions were centrifuged and the resulting supernatants were collected, whereby ATP was extracted from each tissue. Because the ATP level in each tissue varies depending on the amount of tissue used, ATP level per wet weight of each tissue was calculated.

### (5) Measurement of the Pyruvate Dehydrogenase (PDH) Enzyme Activity in Mouse Liver Tissue

The PDH enzyme activity in the liver was measured using five mice from each group on Day 7 after influenza virus infection. The PDH activity was measured by Pyruvate Dehydrogenase (PDH) Enzyme Activity Microplate Assay Kit MSP18 (MitoSciences Inc.) in accordance with the protocol provided by the manufacturer. The measurement principle is as follows: PDH is immunocaptured onto a microplate, where the reaction that PDH's activation enhances to reduce NAD⁺ to NADH is utilized to react coupling a reporter and the resulting absorbance was measured to determine activity level. Specifically, after pulverizing approximately half of the liver in PBS(−) by a Dounce tissue grinder, the protein amount was measured by BCA assay and adjusted to 23.7 mg/mL. After adjusting the homogenized solution, a microplate was filled with the solution at 800 μg/well in accordance with the protocol. Then, a color reagent was reacted with the solution and the changes in absorbance were measured to obtain the PDH enzyme activity. The activity was expressed as a change in the OD value per minute.

### (6) Results

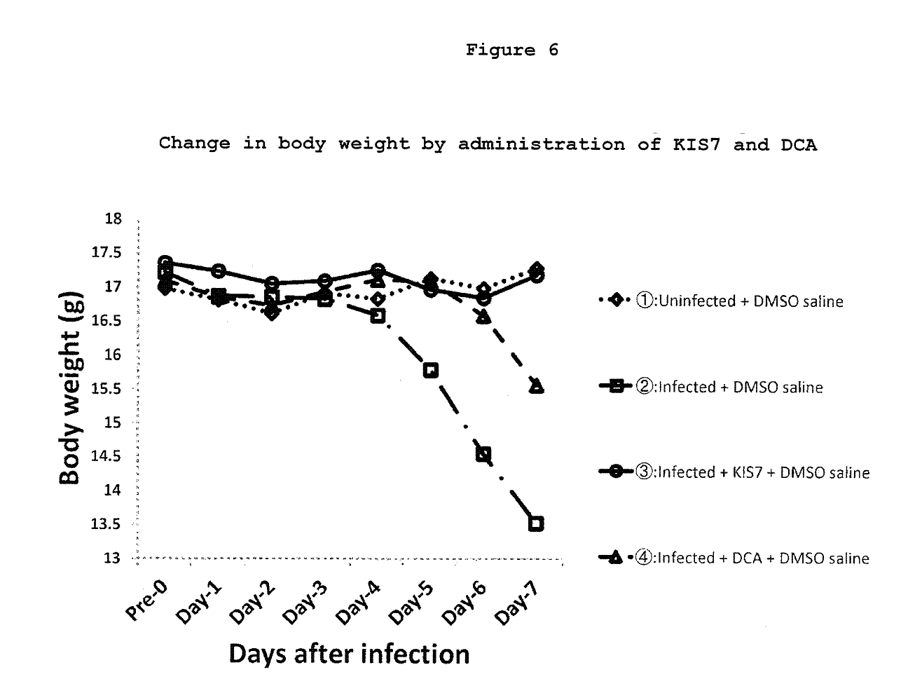

The results of body weight change are shown in FIG. 6. The body weight started to decrease in the group of infected mice administered DMSO only on Day 5 and in the group of DCA administered infected mice on Day 6. In contrast, in the KIS7 administration group, body weight loss was not observed at all, as in the case of non-infected mice. Hence, it was shown that DCA could hardly suppress body weight loss due to influenza infection, whereas KIS7 had a suppressing effect on body weight loss due to influenza infection.

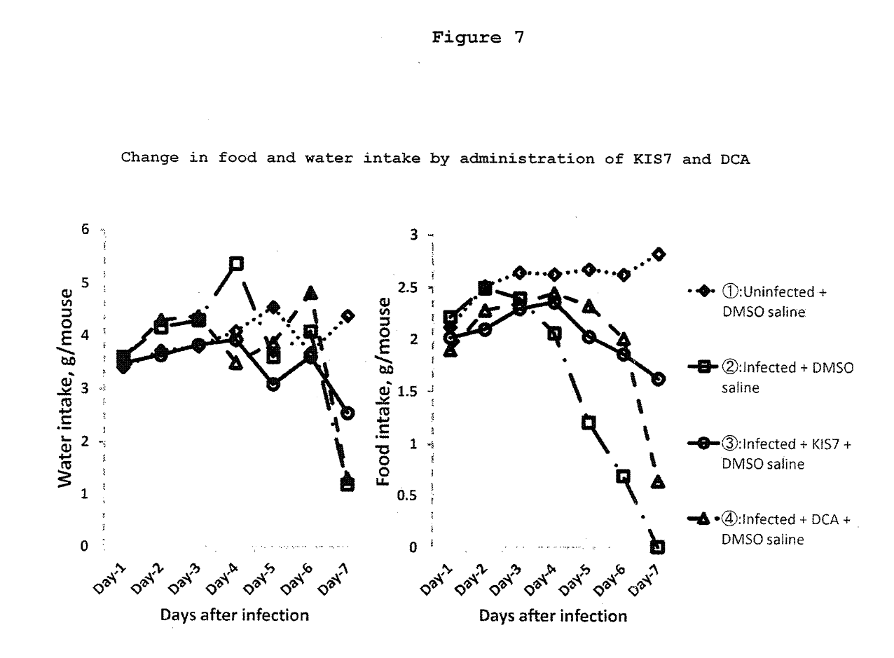

The results of the changes in food and water intake are shown in FIG. 7. With regard to the changes in the amount of water intake as shown on the left in FIG. 7, while the amount of water intake in the group of DCA administered infected mice clearly and markedly decreased on Day 7, a decrease in the amount of water intake was suppressed in the KIS7 administration group. Also, with regard to the changes in the amount of food intake as shown on the right in FIG. 7, while the amount of food intake in the group of DCA administered infected mice started to drastically decrease on Day 6, a decrease in the amount of food intake was suppressed in the KIS7 administration group. Hence, KIS7 was shown to have a suppressing effect on a decrease in the amount of food and water intake due to influenza infection.

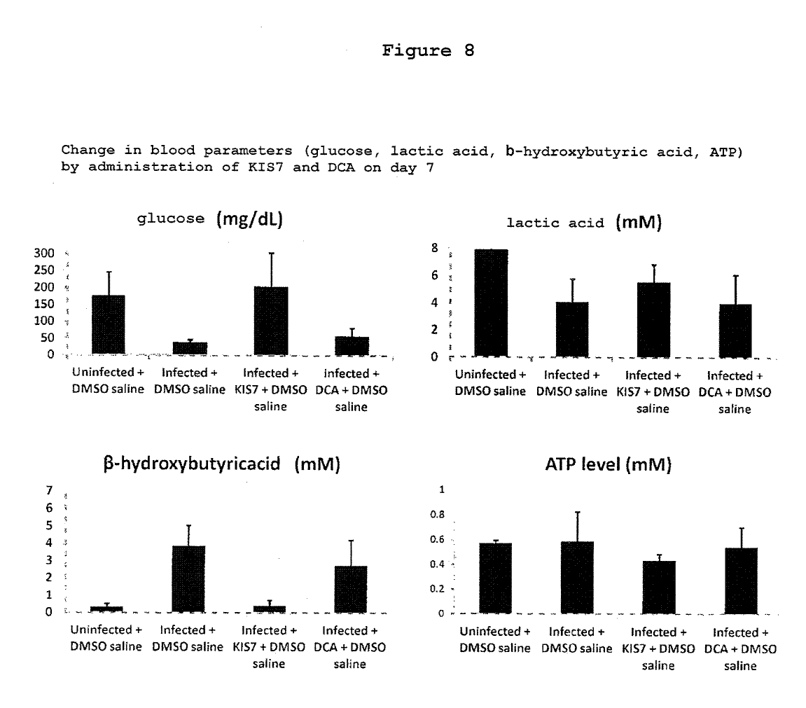

The results of measurement of mouse blood parameters on Day 7 after influenza virus infection are shown in FIG. 8. For all blood parameters, the KIS7 administration group showed equivalent values to non-infected mice. Particularly, as to the blood glucose level and β-hydroxybutyric acid level, the DCA administration group hardly showed any improvement, whereas the KIS7 administration group showed equivalent values to the non-infected group.

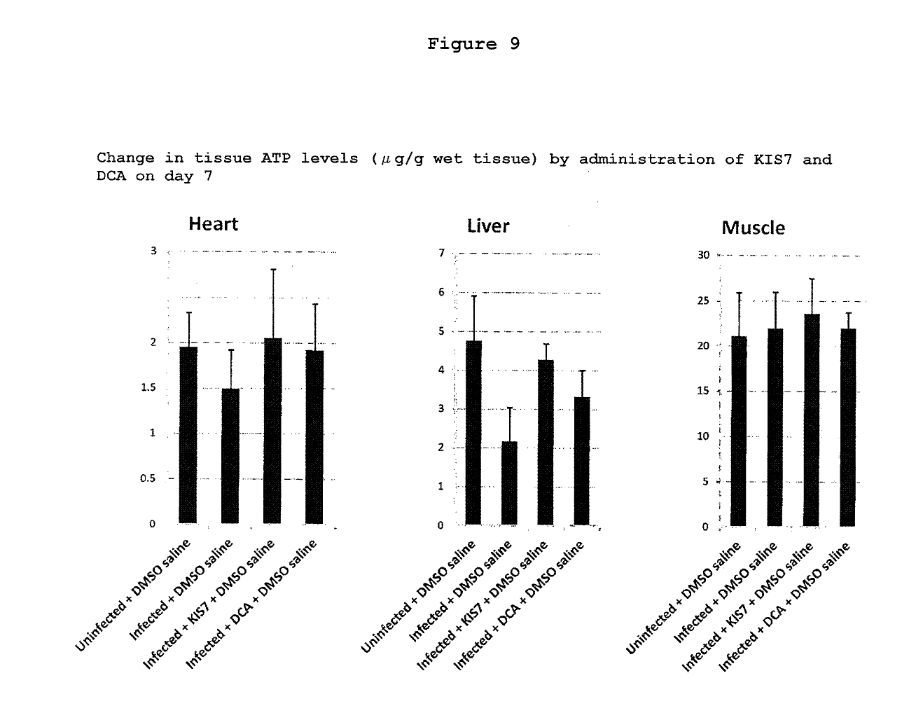

The results of measurement of the ATP level in each mouse tissue on Day 7 after influenza virus infection are shown in FIG. 9. In the heart and liver, the KIS7 administration group showed equivalent ATP levels to non-infected mice, revealing that a decrease in ATP due to influenza virus infection was suppressed.

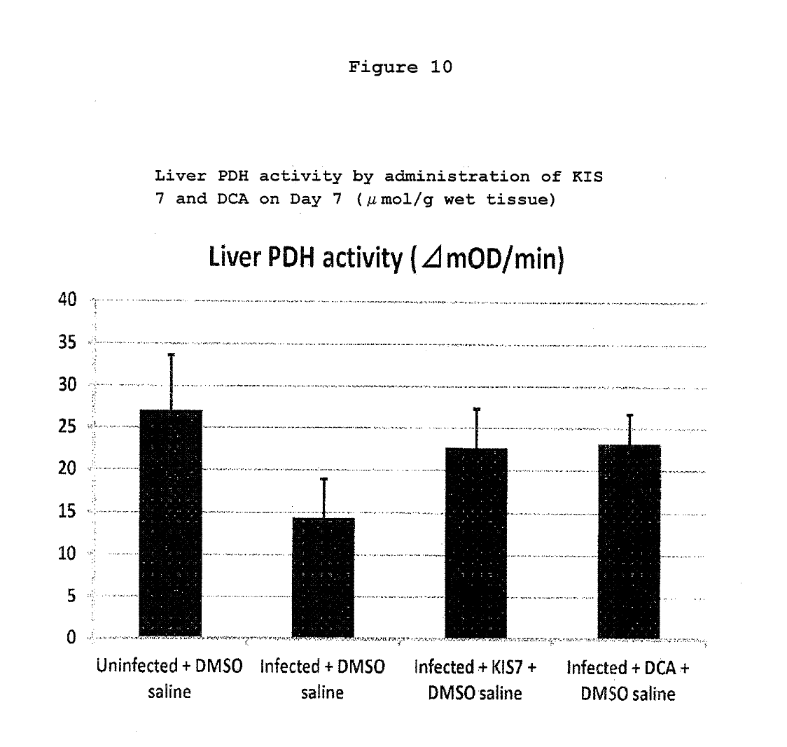

The results of measurement of the PDH enzyme activity in mouse liver tissue on Day 7 after influenza virus infection are shown in FIG. 10. The KIS7 administration group and the DCA administration group were confirmed to have shown equivalent PDH activities to non-infected mice, revealing that KIS7 and DCA suppressed a decrease in PDH activity due to influenza virus infection.

### Example 3

### Effects of KIS7, KIS37, and KIS24 in the Mouse Model of Influenza Infection (14-Day Administration)

The mouse and virus used were the same as those in Example 2. As the KIS compounds, experiments were performed on KIS7, KIS37 and KIS24. Infection and administration were performed by a similar method of Example 2, except for setting the doses of KIS24 and KIS37 at 1.3 mg/kg and 1.6 mg/kg, respectively. The mouse body weight at the time of infection ranged from 15.8 to 17.8 g. Administration was continued for 14 days after virus infection, during which the survival rate, body weight, and the amount of food and water intake were measured in a similar manner to Example 2. Also, the survival rate was measured during 14 days after virus infection.

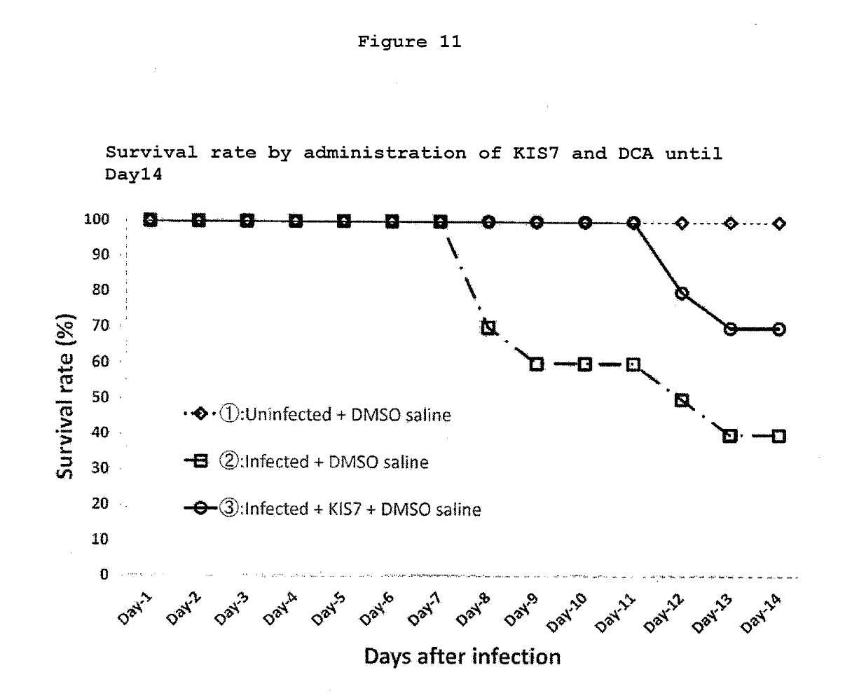

The results of the survival rate during 14 days after influenza infection with administration of KIS7, KIS24, and KIS37 are shown in FIGS. 11, 12, and 13, respectively. In the non-KIS compound-administered groups, the mouse started to die on Day 8, and half or more mice died by Day 14. In contrast, the KIS7 administration group maintained a 100% survival rate until Day 11, even achieving a 70% survival rate as of Day 14. Also, although death occurred on Day 6 in the KIS24 administration group, a 90% survival rate was achieved as of Day 14. Further, the KIS37 administration group maintained a 100% survival rate until Day 10, even maintaining an 80% survival rate as of Day 14.

Also, in KIS24, and KIS37 administration groups, similar improvement effects on body weight and amount of food and water intake were observed as observed in KIS7 administration group (data not shown). Accordingly, KIS7, KIS24, and KIS37 were shown to improve body weight loss, eating and water intake disorder, various parameters accompanied in influenza infection as well as to increase a survival rate by suppressing death.

### Example 4

### Effects of KIS7, KIS24, and KIS37 on Anchorage-Independent Cancer Cell Growth

### (1) Experimental Method

A conventional method was employed for a soft agar colony formation assay of cancer cells HeLaS3 (C. Oneyama et. al. Genes to Cells 2008; 13: 1 to 129). The HeLaS3 cells (4×10⁴) were mixed with 3 ml of soft agar medium (Dulbecco modified Eagle's medium, 10% calf serum, 0.36% agar) and poured into a well of 6 cm-cell culture plate, which was added 5 ml of the base agar medium (Dulbecco modified Eagle's medium, 10% calf serum, 0.7% agar) in advance. After eight days of culture in a carbon dioxide gas incubator at 37° C., colonies were stained with MTT (3-(4,5-dimethylthiazol-2-yl)-2,5-diphenyltetrazolium bromide).

### (2) Results

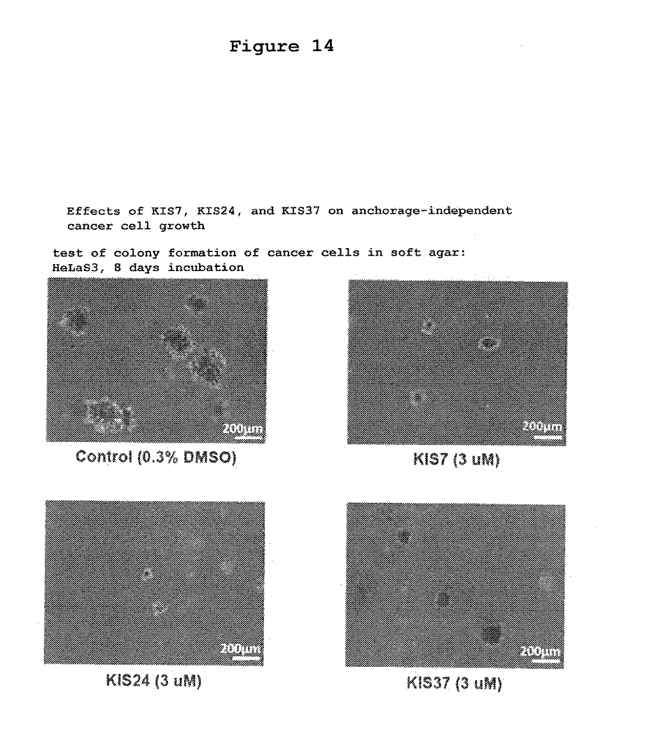

The effects of KIS7, KIS24, and KIS37 on anchorage-independent cancer cell growth are shown in FIG. 14. KIS7, KIS37 and KIS24 inhibited the colony formation of cancer cells HeLaS3 in soft agar at 3 which was of the same order as the inhibition of PDK4.

1.-12. (canceled)

13. A PDK4 inhibitor comprising a compound represented by any one of the following formulaes (I) or pharmaceutically acceptable ester derivatives thereof, or pharmaceutically acceptable salts thereof as an active ingredient: 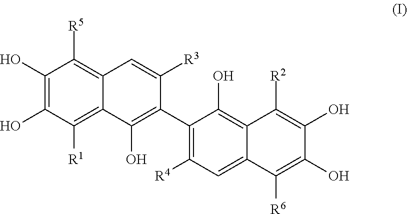
[wherein, R¹ and R² can be the same or different and each represent a formyl group or a 2-carboxyphenyliminomethyl group, and R³ to R⁶ can be the same or different and each represents a linear or branched C1-6 alkyl group].

14. A PDK4 inhibitor comprising a compound represented by any one of the following formulaes (II) or pharmaceutically acceptable ester derivatives thereof, or pharmaceutically acceptable salts thereof as an active ingredient: 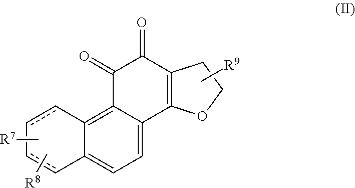
[wherein, a bond represented by a solid line and a broken line represents a single bond or a double bond, R⁷ and R⁸ can be the same or different and each represents a linear or branched C1-6 alkyl group, and R⁹ represents a linear or branched C1-6 alkyl group].

15. A PDK4 inhibitor comprising a compound represented by any one of the following formulaes (III) or pharmaceutically acceptable ester derivatives thereof, or pharmaceutically acceptable salts thereof as an active ingredient: 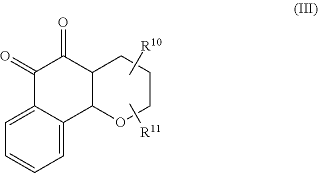
[wherein, R¹⁰ and R¹¹ can be the same or different and each represents a linear or branched C1 to 6 alkyl group].

16. A pharmaceutical composition for diseases or disorders whose development or aggravation is associated with or contributed by the expression or activation of PDK4, comprising a compound represented by any one of the following formulaes (I) or pharmaceutically acceptable ester derivatives thereof, or pharmaceutically acceptable salts thereof as an active ingredient: 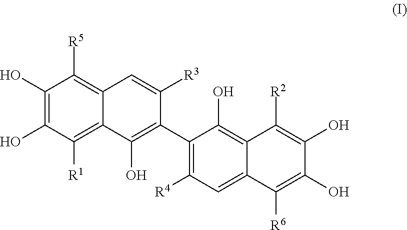
[wherein, R¹ and R² can be the same or different and each represent a formyl group or a 2-carboxyphenyliminomethyl group, and R³ to R⁶ can be the same or different and each represents a linear or branched C1-6 alkyl group].

17. A pharmaceutical composition for diseases or disorders whose development or aggravation is associated with or contributed by the expression or activation of PDK4, comprising a compound represented by any one of the following formulaes (II) or pharmaceutically acceptable ester derivatives thereof, or pharmaceutically acceptable salts thereof as an active ingredient: 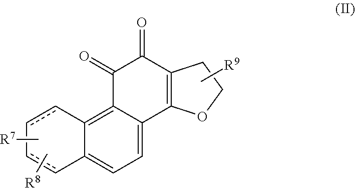
[wherein, a bond represented by a solid line and a broken line represents a single bond or a double bond, R⁷ and R⁸ can be the same or different and each represents a linear or branched C1-6 alkyl group, and R⁹ represents a linear or branched C1-6 alkyl group].

18. A pharmaceutical composition for diseases or disorders whose development or aggravation is associated with or contributed by the expression or activation of PDK4, comprising a compound represented by any one of the following formulaes (III) or pharmaceutically acceptable ester derivatives thereof, or pharmaceutically acceptable salts thereof as an active ingredient: 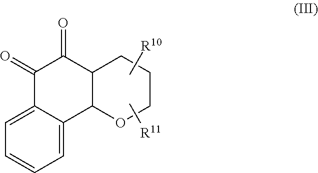
[wherein, R¹⁰ and R¹¹ can be the same or different and each represents a linear or branched C1 to 6 alkyl group].

19. The pharmaceutical composition of claim 16, wherein said diseases or disorders whose development or aggravation is associated with or contributed by the expression or activation of PDK4 is aggravation after influenza infection.

20. The pharmaceutical composition of claim 16, wherein said diseases or disorders whose development or aggravation is associated with or contributed by the expression or activation of PDK4 is loss of appetite.

21. The pharmaceutical composition of claim 16, wherein said diseases or disorders whose development or aggravation is associated with or contributed by the expression or activation of PDK4 is mitochondrial disease, or a disease or disorder associated with reduced ATP production.

22. The pharmaceutical composition of claim 16, wherein said diseases or disorders whose development or aggravation is associated with or contributed by the expression or activation of PDK4 is diabetes.

23. The pharmaceutical composition of claim 16, wherein said diseases or disorders whose development or aggravation is associated with or contributed by the expression or activation of PDK4 is cancer.

24. A cosmetic composition comprising a compound represented by any one of the following formulaes (I) to (III) or pharmaceutically acceptable ester derivatives thereof, or pharmaceutically acceptable salts thereof as an active ingredient: 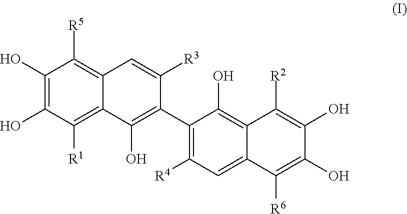
[wherein, R¹ and R² can be the same or different and each represent a formyl group or a 2-carboxyphenyliminomethyl group, and R³ to R⁶ can be the same or different and each represents a linear or branched C1-6 alkyl group]; 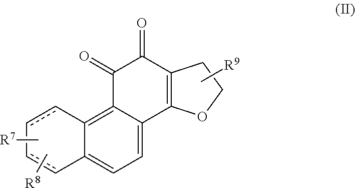
[wherein, a bond represented by a solid line and a broken line represents a single bond or a double bond, R⁷ and R⁸ can be the same or different and each represents a linear or branched C1-6 alkyl group, and R⁹ represents a linear or branched C1-6 alkyl group]; 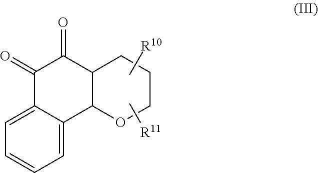
[wherein, R¹⁰ and R¹¹ can be the same or different and each represents a linear or branched C1 to 6 alkyl group].

25. The pharmaceutical composition of claim 17, wherein said diseases or disorders whose development or aggravation is associated with or contributed by the expression or activation of PDK4 is aggravation after influenza infection.

26. The pharmaceutical composition of claim 18, wherein said diseases or disorders whose development or aggravation is associated with or contributed by the expression or activation of PDK4 is aggravation after influenza infection.

27. The pharmaceutical composition of claim 17, wherein said diseases or disorders whose development or aggravation is associated with or contributed by the expression or activation of PDK4 is loss of appetite.

28. The pharmaceutical composition of claim 18, wherein said diseases or disorders whose development or aggravation is associated with or contributed by the expression or activation of PDK4 is loss of appetite.

29. The pharmaceutical composition of claim 17, wherein said diseases or disorders whose development or aggravation is associated with or contributed by the expression or activation of PDK4 is mitochondrial disease, or a disease or disorder associated with reduced ATP production.

30. The pharmaceutical composition of claim 18, wherein said diseases or disorders whose development or aggravation is associated with or contributed by the expression or activation of PDK4 is mitochondrial disease, or a disease or disorder associated with reduced ATP production.

31. The pharmaceutical composition of claim 17, wherein said diseases or disorders whose development or aggravation is associated with or contributed by the expression or activation of PDK4 is diabetes.

32. The pharmaceutical composition of claim 18, wherein said diseases or disorders whose development or aggravation is associated with or contributed by the expression or activation of PDK4 is diabetes.
